(19) United States
(12) Patent Application Publication
(10) Pub. No.: US 2007/0264640 A1
(43) Pub. Date: Nov. 15, 2007

(54) Array-based assays using split-probe nucleic acid arrays

(76) Inventors: Michael T. Barrett, Mountain View, CA (US); Nicholas M. Sampas, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/433,876

(22) Filed: May 12, 2006

(51) Int. Cl. C12Q 1/68; C12M 3/00

(52) U.S. Cl. 435/6; 435/287.2

(57) ABSTRACT

Methods and compositions for performing array-based assays of a nucleic acid sample, such as genomic sample assays, e.g., comparative genomic (aCGH) assays, are provided. Aspects of the invention include arrays containing split-probe nucleic acids that include two linked domains that flank a nucleic acid region, such as a genomic region, of interest. The subject methods and compositions find use in a number of applications, including aCGH applications, e.g., identifying genomic copy number, evaluating the methylation status, etc. Also provided are kits that include the subject arrays.

### BACKGROUND OF THE INVENTION

Many genomic and genetic studies are directed to the identification of differences in gene dosage or expression among cell populations for the study and detection of disease. For example, many malignancies involve the gain or loss of DNA sequences resulting in activation of oncogenes or inactivation of tumor suppressor genes. In addition, changes in genomic DNA methylation can impact the expression of oncogenes and tumor suppressors. Furthermore, alterations in DNA methylation patterns are associated with a variety of non-neoplastic diseases and developmental disorders. Thus identification of the genetic and epigenetic events in normal and abnormal cell types and tissues can facilitate efforts to define the biological basis for disease and development, develop predictors of disease outcomes, improve prognosis of therapeutic response, and permit earlier disease detection.

Comparative genomic hybridization (CGH) is one approach that has been employed to detect the presence and identify the location of amplified or deleted genomic sequences. In one implementation of CGH, genomic DNA is isolated from normal reference cells, as well as from test cells (e.g., tumor cells). The two nucleic acid samples are differentially labeled and then simultaneously hybridized in situ to metaphase chromosomes of a reference cell. Chromosomal regions in the test cells which are at increased or decreased copy number relative to the reference cells can be identified by detecting regions where the ratio of the signals from the two distinguishably labeled nucleic acids is altered. For example, those regions that have been decreased in copy number in the test cells will show relatively lower signal from the test nucleic acid sample than the reference compared to other regions of the genome. Regions that have been increased in copy number in the test cells will show relatively higher signal from the test nucleic acid sample.

Genomic DNA methylation is found in a wide variety of organisms, including bacteria, animals, plants and fungi and plays a role in many cellular processes. For example, in prokaryotic organisms, DNA methylation coordinates DNA replication and the cell cycle, helps distinguish self from nonself DNA (e.g, the restriction/modification system), and can direct post-replication mismatch repair. In eukaryotic organisms, genomic DNA methylation impacts the regulation of gene transcription/expression, X chromosome inactivation and the regulation of development (e.g., genomic imprinting). As such, an understanding of the methylation status of genomic DNA is central for understanding fundamental aspects of cellular growth, development, and neoplasias (see Jeltsch, A. Chembiochem 2002, 3:274-293 for review).

For example, the human genome is estimated to contain 50×10⁶ CpG dinucleotides, the predominant sequence recognition motif for mammalian DNA methyltransferases which methylate the cytosine at position C-5. Clusters of CpGs, or “CpG islands”, are present in the promoter or intronic regions of approximately 40% of mammalian genes (Larsen et al., Genomics (1992) 13:1095-1107). Methylation of cytosine residues contained within CpG islands (i.e. “CpG island methylation”) has generally been correlated with reduced gene expression, and is thought to play a fundamental role in many mammalian processes, including embryonic development, X-inactivation, genomic imprinting, regulation of gene expression, and host defense against parasitic sequences, as well as abnormal processes such as carcinogenesis, fragile site expression, and cytosine to thymine transition mutations. In addition, alterations in methylation levels of CpGs occur under different physiologic and pathologic conditions. Accordingly, CpG methylation is an area of intense interest to the scientific community.

Methods to evaluate genomic DNA methylation status have been developed (Proc. Natl. Acad. Sci. (1992) 89: 1827-1831; Huang et al., Human Mol. Genet. 8, 459-70, 1999; WO 02/086163 A1; Plass et al., Genomics 58:254-62, 1999; Gonzalgo et al., Cancer Res. 57: 594-599, 1997; and Toyota et. al., Cancer Res. 59: 2307-2312, 1999). However, these techniques are unsuitable as high-throughput tools for investigating genomic DNA methylation because they generally require a number of amplification steps or chemical treatments that can lead to unreliable results.

In addition, in conventional methods for evaluating genomic copy number variations and methylation status, designing genomic probes with favorable hybridization characteristics to certain genomic regions of interest can be difficult. For example, designing oligonucleotide probes specific for CpG islands in mammalian DNA is difficult, if not impossible, due to the repetitive nature of the target sequences, as well as the significant secondary structure and high melting temperature (Tₘ) of the target sequences.

Accordingly, while several methods have proved successful in evaluating genomic copy number or methylation of a genomic region of interest, such methods are unsuitable for all genomic regions of interest. As such, a great need still exists for reliable, straightforward and high-throughput methods for the evaluation of genome copy number, particularly for using in evaluating methylation status of a genomic sample. This invention meets this, and other, needs.

### Relevant Literature

Literature of interest includes: Jeltsch, A. (Chembiochem (2002) 3:274-293), Laird, P. W. (Nat Rev Cancer (2003) 3:253-66), Fraga, M. F. and M. Esteller (Biotechniques (2002) 33:632-49), Oakeley, E. J. (Pharmacol Ther. (1999) 84:389-400), Herman, J. G. et al., (Proc Natl Acad Sci. (1996) 93:9821-6), Costello. J. F., et al., (Nat Genet (2000) 24:132-8), Zardo, G. et al., (Nat Genet (2002) 32:453-8), Kutyavin, I. V. et al. (Biochemistry (1996) 35:11170-6), Huang et al., (Human Mol. Genet. (1999) 8: 459-70), Plass et al., (Genomics (1999) 58:254-62), Gonzalgo et al., (Cancer Res. (1997) 57: 594-599), Toyota et. al., (Cancer Res. (1999) 59: 2307-2312), Cottrell et al., (Ann N Y Acad Sci. (2003) 983:120-130), Gitan et al., (Genome Research (2003) 12:158-164), Kutyavin et al., (Nucl. Acids Res. (2002) 30: 4952-4959), Takai et al., (Proc. Natl. Acad. Sci. (2002) 99:3740-3745); Strichman-Almashanu et al., (Genome Research (2002) 12:543-554); Sved et al., (Proc. Natl. Acad. Sci. (1990) 87:4692-6), Antequera et al., Proc. Natl. Acad. Sci. (1993) 90:11995-9 and Chen et al., (Am. J. Pathol. (2003) 163:37-45); published U.S. Patent Applications 20030211474, 20030215842, 20030186250, 20020123053, 20030129602 and 20020006623; and PCT publication WO 02/086163.

### SUMMARY OF THE INVENTION

Methods and compositions for performing array-based assays, such as genomic assays, e.g., comparative genomic (aCGH) assays, are provided. Specifically, the invention provides arrays containing split-probe nucleic acids, e.g., that comprise two linked domains that flank a genomic region of interest. The subject methods and compositions find use in a number of aCGH applications, including identifying genomic copy number, evaluating methylation status, etc. Also provided are kits that include the subject arrays.

### BRIEF DESCRIPTION OF THE FIGURES

FIG. 1 provides a schematic diagram showing the split-probe nucleic acid of the invention and its hybridization characteristics with cleaved and uncleaved target nucleic acids.

FIG. 2 provides a plot of the mean signals from sets of split-probes as a function of the length of the loops in their respective targets.

FIG. 3 provides the slope response for the chromosome X split-probes (3307 probes, corresponding to 369 distinct targets) in aCGH assays comparing 46,XY (male) and 46,XX (female) samples.

### DEFINITIONS

The term “nucleic acid” and “polynucleotide” are used interchangeably herein to describe a polymer of any length, e.g., greater than about 10 bases, greater than about 100 bases, greater than about 500 bases, greater than 1000 bases, usually up to about 10,000 or more bases composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally occurring nucleotides include nucleobases chosen from guanine, cytosine, adenine and thymine (G, C, A and T, respectively).

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length. Oligonucleotides are usually synthetic and, in many embodiments, are fewer than 70 nucleotides in length.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other nucleic acids that are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “surface-bound polynucleotide” refers to a polynucleotide that is immobilized on a surface of a solid substrate, where the substrate can have a variety of configurations, e.g., a sheet, bead, or other structure. In certain embodiments, the collections of split-probe oligonucleotides employed herein are present on a surface of the same planar support, e.g., in the form of an array.

The phrase “labeled population of nucleic acids” refers to a mixture of nucleic acids that are detectably labeled, e.g., fluorescently labeled, such that the presence of the nucleic acids can be detected by assessing the presence of the label. A labeled population of nucleic acids is “made from” a “genomic composition” or a “sample composition.” the composition is usually employed as a template for making the population of nucleic acids.

The term “array” encompasses the term “microarray” and refers to an ordered array presented for binding to nucleic acids and the like.

An “array,” includes any two-dimensional or substantially two-dimensional (as well as a three-dimensional) arrangement of spatially addressable regions bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm ², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse-jets of either precursor units (such as nucleotide or amino acid monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different moieties (e.g., different oligonucleotides each having a unique sequence) such that a region (i.e., a “feature” or “spot” of the array) at a particular predetermined location (i.e., an “address”) on the array will detect a particular sequence in a sample. Array features are typically, but need not be, separated by intervening spaces. In the case of an array in the context of the present application, the “population of labeled nucleic acids” or “sample composition” and the like will be referenced as a moiety in a mobile phase (typically fluid), to be detected by “surface-bound polynucleotides” which are bound to the substrate at the various regions. These phrases are synonymous with the arbitrary terms “target” and “probe”, or “probe” and “target”, respectively, as they are used in other publications.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if intervening areas that lack features of interest are present.

An “array layout” refers to one or more characteristics of the features, such as feature positioning on the substrate, one or more feature dimensions, and an indication of a moiety at a given location. “Hybridizing” and “binding” , with respect to nucleic acids, are used interchangeably.

The term “stringent assay conditions” as used herein refers to conditions that are compatible to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. The term stringent assay conditions refers to the combination of hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions determines whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equilvalent hybridization and wash conditions and for reagents and buffers, e.g., SSC buffers and equivalent reagents and conditions.

A specific example of stringent assay conditions is rotating hybridization at 65° C. in a salt based hybridization buffer with a total monovalent cation concentration of 1.5M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent hybridization conditions may also include a “prehybridization” of aqueous phase nucleic acids with complexity-reducing nucleic acids to suppress repetitive sequences and reduce the complexity of the sample prior to hybridization. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct. In other words, a mixture is not addressable. To be specific, an array of surface-bound polynucleotides, as is commonly known in the art and described below, is not a mixture of surface-bound polynucleotides because the species of surface-bound polynucleotides are spatially distinct and the array is addressable.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are wellknown in the art and include, for example, ion-exchange chromatography, affinity chromatography and sedimentation according to density. Generally, a substance is purified when it exists in a sample in an amount, relative to other components of the sample that is not found naturally.

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent. Additionally, the “binding characteristic” of a target to a probe means the result of measuring the amount of target associated with a probe after contacting the target (or target sample) to a probe.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

If a subject split-probe nucleic acid “corresponds to”, “is designed for” or is “specific for” a certain genomic region, the split-probe nucleic acid usually comprises a first domain and a second domain that flank a genomic domain that contains the genomic region of interest. As such, the genomic domain flanked by the first and second domains of the split probe nucleic acid is at least the size of the genomic region of interest and, in many embodiments, is larger than the genomic region of interest.

### DETAILED DESCRIPTION

As stated above, methods and compositions for performing array-based assays, such as genomic assays, e.g., comparative genomic (aCGH) assays, are provided. Specifically, the invention provides arrays containing split-probe nucleic acids, e.g., that comprise two linked domains that flank a genomic region of interest.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates that may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

As summarized above, the present invention provides split-probe nucleic acid arrays and methods for using the same. In further describing the present invention, the subject arrays are first described in greater detail, followed by more in-depth review of representative methods of using the arrays, as well as representative applications in which subject invention finds use. Finally, representative kits for use in practicing the subject methods will be described.

### Split-Probe Arrays

As summarized above, the subject invention provides split-probe arrays. An aspect of the subject split probe arrays is the presence of at least one feature on the array that includes a split-probe nucleic acid. The split-probe nucleic acids of the subject arrays are specific for a target genomic domain, i.e., genomic region of interest. By target genomic domain is meant genomic location or region whose presence, and in many embodiments copy number, is of interest and is to be assayed. In certain embodiments, the target genomic domain is a domain that is not readily directly detectable with array bound nucleic acid, e.g., because the target genomic region has a sequence that makes it difficult to directly assay using a surface immobilized probe nucleic acid. The target genomic domain (i.e., the genomic region flanked by the 5′ and 3′ domains of the split-probe nucleic acid) can be of virtually any size. In certain embodiments, the target genomic domain can be up to 5000 nucleotides in length, including from about 4 to about 1000, from about 5 to about 500, as well as from about 10 to about 250 nucleotides in length. In certain embodiments, the target genomic domain contains a putative methylation site, where in certain of these embodiments the putative methylation site is located in a recognition sequence of a methylation-sensitive restriction endonuclease. By putative DNA methylation site is meant a site that can be (or is predicted to be) methylated by an enzyme (e.g., methyltransferase). In some of these embodiments (e.g., when the genome is mammalian), the putative methylation site is a CpG site, where the CpG site may be present in a CpG island. In yet other embodiments, the target genomic domain may include repetitive sequences, e.g., that impart to the domain a secondary structure.

As reviewed in greater detail below, certain embodiments of the methods of the invention exploit methylation-sensitive restriction endonucleases coupled with aCGH to assess the methylation status of a genomic region of interest. As such, in certain embodiments, the putative methylation site present in the genomic region of interest falls within a recognition sequence for a methylation-sensitive restriction endonuclease. In certain of these embodiments, the putative methylation site falls within a recognition sequence for a methylation-sensitive restriction endonuclease and a methylation-insensitive isoschizomer. An isoschizomer of a restriction enzyme is one that has the same recognition sequence and cleaves the same site in the same manner. Although they cleave the same recognition sequences, isoschizomers may differ in their activity in the presence of various modifiers of DNA sequences such as methylation. An example of a methylation-sensitive restriction enzyme and its methylation-insensitive isoschizomer is SmaI and XmaI, respectively.

In representative embodiments, the split-probe nucleic acids of the invention include two linked domains, i.e., first and second domains linked to each other, either directly or through a linking group. The first domain corresponds to a 5′ genomic region that is 5′ of a target genomic domain. By corresponds is meant that the first domain has a sequence that specifically binds to either 5′ genomic region or a complementary sequence thereof. For example, the first domain may hybridize to a nucleic acid having a sequence of the 5′ genomic sequence (or complement thereof) under stringent conditions.

The second domain corresponds to a 3′ genomic region that is 3′ of the target genomic domain. As above, by corresponds is meant that the second domain has a sequence that specifically binds to either 3′ genomic region or a complementary sequence thereof. For example, the second domain may hybridize to a nucleic acid having a sequence of the 3′ genomic sequence (or complement thereof) under stringent conditions.

As such, the 5′ and 3′ domains of the split-probe nucleic acid flank the target genomic domain. The designation of the flanking domains as 5′ and 3′ to the target genomic domain merely indicates that the genomic domains are linked to each other in such a way as to maintain their relative orientation with regard to the genomic domain. In other words, the sequences of the 5′ and 3′ domains have sequences found in the same strand of the genomic DNA and the 3′ end of the 5′ domain is linked to the 5′ end of the 3′ domain. As such, this designation is in no way meant to impart any limitation on the order of the domains in the split-probe oligonucleotide with regard to the orientation of elements present in the genomic domain (e.g., with respect to the orientation of a transcribed gene).

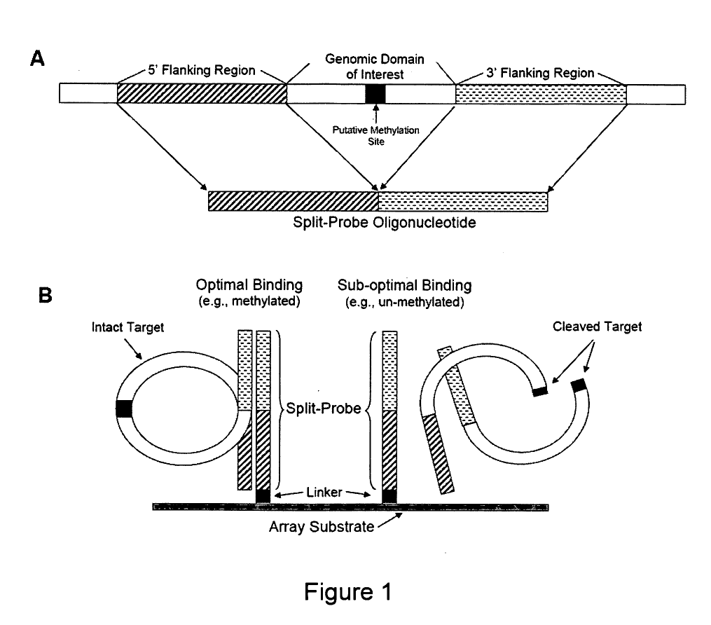

As demonstrated in the Experimental section below and shown in FIG. 1, the split-probe nucleic acids of the present invention specifically bind to target nucleic acids containing the target genomic domain when flanked by sequences at its 5′ and 3′ ends to which the 5′ and 3′ domains of the split probe nucleic acid specifically bind, e.g., under stringent hybridization conditions.

In certain embodiments of the present invention, each domain of a split probe nucleic acid (i.e., the 5′ and 3′ domains) is from about 5to about 100 nucleotides in length, such as from about 10 to about 50 nucleotides, and including from about 15 to about 40 nucleotides, and from about 20 to about 30 nucleotides in length. As such, the total length of a split-probe nucleic acid of these embodiments of the subject invention is from about 10 to about 200 nucleotides, such as from about 20 to about 100 nucleotides, and including from about 30 to about 80 nucleotides, and from 40 to about 60 nucleotides.

In certain embodiments, split-probe nucleic acids of the invention can include a linker domain, which can be of varying lengths, and serves to link the split-probe nucleic acid to a surface (e.g., a solid support). In representative embodiments, this linker domain, if present, may range in length from about 10 to about 50 nucleotides in length, such as from about 15 to about 40 nucleotides, and including from about 20 to about 30 nucleotides and may have any convenient sequence. In certain embodiments, all of the distinct split probe nucleic acids displayed on the surface of the array have a common linker domain linking the nucleic acids to the surface of the array.

In certain embodiments, an additional set of control sequences are used to eliminate the contribution to the signal that is due to the hybridization of the target(s) exclusively to an individual domain of the split probe. Each of these control sequences consists of a sequence that is identical to one of the domains of the split probe. In the case of the distal domain, the sequence is supported by a linker sequence that is substantially identical in length as the proximal region of the split probe, thus allowing the probe to be nominally the same distance from the surface and experience the same environmental effects. This linker sequence is a non-binding sequence (or negative control sequence) that is known to be unstructured and hence to not bind specifically to any domain within the genome of the specimen under test. Alternatively, the linker could be another abasic nucleic acid backbone polymer or any other molecule of nominally the same length as the proximal domain of the split probe sequence. In this embodiment, each split probe sequence would have two control probe sequences, one to control for binding to one (3′ or proximal) domain of the split probe and another to control for the other (5′ or distal) domain of the split probe sequence.

It is to be understood that the 5′ and 3′ domains of the split-probe oligonucleotides of the invention need not have the same number of nucleotides. Indeed, in many embodiments, the 5′ and 3′ domains have different numbers of nucleotides. Rather, in certain embodiments, the hybridization characteristics of the 5′ and 3′ domains are of interest (e.g., melting temperature, base composition, secondary structure predictions, etc.), such that all of the probes have similar, i.e., substantially identical, hybridization characteristics. In certain of these embodiments, the 5′ and 3′ domains can have similar hybridization properties, either predicted or empirically determined. In certain embodiments, all of the split-probe nucleic acids have similar hybridization characteristics as determined using a test nucleic acid composition that contains the same amount of each probe displayed on the array. In certain embodiments, any difference in detected signal from any two features of the array does not reproducibly vary by more than about 80%, such as by no more than about 50%, e.g., under the hybridization conditions described in the Experimental section below.

In representative embodiments, the split-probe nucleic acids of the arrays employed in the present invention include a 5′ and a 3′ domain with similar hybridization properties that flank a genomic region of interest, where the genomic region of interest has a putative methylation site present in a recognition sequence for a methylation-sensitive restriction enzyme.

In certain embodiments of the invention, the split-probe nucleic acids are “surface-bound split-probe nucleic acids”, where such nucleic acid is bound, usually covalently but in certain embodiments non-covalently, to a surface of a solid substrate or support, i.e., a sheet, bead, or other substrate or structure. In certain embodiments, surface-bound split-probe nucleic acids may be immobilized on a surface of a planar support, e.g., as part of an array.

A “split-probe nucleic acid feature” is a feature of an array, i.e., a spatially addressable area of an array, as described above, which contains a plurality of surface-bound split-probe nucleic acids. Accordingly, a feature contains “surface-bound” nucleic acids that are bound, in some embodiments covalently, to an area of substrate surface. In representative embodiments, a single type of nucleic acid is present in each split-probe nucleic acid feature (i.e., all the nucleic acids in the feature have the same sequence). However, in certain embodiments, the nucleic acids in a given feature may be a mixture of distinct nucleic acids of differing sequence.

The subject arrays may contain a single split-probe nucleic acid feature. However, in many embodiments, the subject arrays contain more than one such feature, and those features may correspond to (i.e., may be used to detect) a plurality of genomic regions of a genome. Accordingly, the subject arrays may contain a plurality of features (i.e., 2 or more, about 5 or more, about 10 or more, about 15 or more, about 20 or more, about 30 or more, about 50 or more, about 100 or more, about 200 or more, about 500 or more, about 1000 or more, up to about 40,000 or about 500,000 or more features, etc.), each containing different split-probe nucleic acids. In certain embodiments, therefore, the subject arrays contain a plurality of subject split-probe nucleic acid features that correspond to a plurality of genomic regions of a genome.

For example, in embodiments in which CpG methylation in CpG islands is being assessed, the subject arrays may contain split-probe nucleic acid features for, i.e., corresponding to, all of the predicted CpG islands of a particular genome. The subject arrays for investigating methylation status of human CpG islands may therefore contain up to about 500,000 different split-probe nucleic acid features.

The subject split-probe nucleic acid features are usually present in an array of nucleic acid features. In certain embodiments, at least one of the nucleic acid features includes a split-probe nucleic acid, whereas in other embodiments more than one feature comprises a split-probe nucleic acid. In certain embodiments in which multiple features include split-probe nucleic acids, the split-probe nucleic acids of distinct features are specific for a different genomic region. In general, arrays suitable for use in performing the subject methods contain a plurality (i.e., at least about 100, at least about 500, at least about 1000, at least about 2000, at least about 5000, at least about 10,000, at least about 20,000, usually up to about 100,000 or more) of addressable features, where each feature contains a nucleic acid probe (including split-probe nucleic acids) that are linked to a solid support, e.g., a planar surface thereof.

In certain embodiments, the subject arrays may include features of other nucleic acids, such as other oligonucleotides, or other cDNAs, or inserts from phage, BACs or plasmid clones. As such, while the subject arrays usually contain features of split-probe nucleic acids, they may also contain features of other non-split probe nucleic acids, e.g., that are about 101-5000 bases in length, about 5001-50,000 bases in length, or about 50,001-200,000 bases in length, depending on the platform used. If other polynucleotide features are present on a subject array, they may be interspersed with, or in a separately-hybridizable part of the array from, the subject oligonucleotides.

In certain embodiments, genomic regions of interest are represented on a given array (where by representative is meant that the genomic region of interest corresponds to one or more features on an array) by at least about 2, about 5, or about 10 or more, including up to about 20 or more split-probe features. As such, the subject arrays of these embodiments include one or more sets of split-probe features, where a given set includes two or more different split probes that correspond to the same target genomic domain. The feature of a given set may contain probes of different, non-overlapping, or, in some embodiments, overlapping, sequence. In other words, a number of distinct split-probe nucleic acids can have overlapping intervening genomic domains. For example, a first split probe nuclei acid can have 5′ and 3′ domains that flank a 50 nucleotide intervening genomic domain and a second split probe nucleic acid can have 5′ and 3′ domains that flank a 100 nucleotide intervening genomic domain that includes the intervening genomic domain of the first split-probe oligonucleotide. The overlap between the intervening genomic regions can be of any number of nucleotides and need not contain all of one of the genomic regions. For example, the 50 nucleotide intervening genomic domains of two split-probe nucleic acids illustrated above may overlap by only 15 nucleotides.

In certain embodiments, a set of split-probe oligonucleotides specific for a particular genomic region of interest can be present on the array. In these embodiments, the set of split-probes can each share a common 5′ domain while having a different 3′ domain (or vice versa). Each of the 3′ domains correspond to a 3′ region of the genomic target nucleic acid that is successively further from the 5′ domain, making the intervening genomic domain for each split-probe in the set successively larger.

The subject split-probe oligonucleotide arrays can be fabricated using any means, including drop deposition from pulse jets or from fluid-filled tips, etc, or using photolithographic means. Either polynucleotide precursor units (such as nucleotide monomers), in the case of in situ fabrication, or previously synthesized polynucleotides (i.e., split-probe oligonucleotides) can be deposited. Such methods are described in detail in, for example U.S. Pat. Nos. 6,242,266, 6,232,072, 6,180,351, 6,171,797, 6,323,043, etc., the disclosures of which are herein incorporated by reference.

### Methods

Also provided are methods of using the subject arrays, e.g., in aCGH applications. Array-based CGH (aCGH) assays may be performed in a number of ways. In representative embodiments, the first step is labeling a nucleic acid composition, e.g., a sample or genomic source to be assayed, to make labeled populations of nucleic acids which may be distinguishably labeled, contacting the labeled populations of nucleic acids with at least one array of surface bound nucleic acids under specific hybridization conditions, and analyzing any data obtained from hybridization of the nucleic acids of the sample to the surface bound nucleic acids. Such methods are generally well known in the art (see, e.g., Pinkel et al., Nat. Genet. (1998) 20:207-211; Hodgson et al., Nat. Genet. (2001) 29:459-464; Wilhelm et al., Cancer Res. (2002) 62: 957-960) and, as such, need not be described herein in any great detail.

The sample may be any of a variety of different samples, where in representative embodiments the sample is a genomic sample, and may be referred to as a genomic source. By “genomic source” is meant the initial nucleic acids that are used as the original nucleic acid source from which the solution phase nucleic acids employed in a given aCGH assay are produced, e.g., as a template in the labeled solution phase nucleic acid generation protocols described in greater detail below.

The genomic source may be prepared using any convenient protocol. In many embodiments, the genomic source is prepared by first obtaining a starting composition of genomic DNA, e.g., a nuclear fraction of a cell lysate, where any convenient means for obtaining such a fraction may be employed and numerous protocols for doing so are well known in the art. The genomic source is, in many embodiments of interest, genomic DNA representing the entire genome from a particular organism, tissue or cell type. However, in certain embodiments, the genomic source may comprise a portion of the genome, e.g., one or more specific chromosomes or regions thereof, such as PCR amplified regions produced with pairs of specific primers, or extrachromosomal elements such as mitochondria, viral particles, plasmids, or double minute chromosome fragments.

A given initial genomic source may be prepared from a subject, for example a plant or an animal. In certain embodiments, the average size of the constituent molecules that make up the initial genomic source typically have an average size of at least about 1 Mb, where a representative range of sizes is from about 50 to about 250 Mb or more, while in other embodiments, the sizes may not exceed about 1 Mb, such that they may be about 1 Mb or smaller, e.g., less than about 500 kb, etc.

In certain embodiments, the subject from which a genomic source is obtained is “mammalian”, where this term is used broadly to describe organisms which are within the class mammalia, including the orders carnivore (e.g., dogs and cats), rodentia (e.g., mice, guinea pigs, and rats), and primates (e.g., humans, chimpanzees, and monkeys), where of particular interest in certain embodiments are human or mouse subjects. In certain embodiments, the genomic source derived from a subject is complex, as the genome of a subject can contain at least about 1×10⁸ base pairs, including at least about 1×10⁹ base pairs, e.g., about 3×10⁹ base pairs.

Where desired, the initial genomic source may be fragmented in the generation protocol, as desired, to produce a fragmented genomic source, where the molecules have a desired average size range, e.g., up to about 10 kb, such as up to about 1 kb, where fragmentation may be achieved using any convenient protocol, including but not limited to: mechanical protocols, e.g., sonication, shearing, etc., chemical protocols, e.g., enzyme digestion, etc.

Where desired, the initial genomic source may be amplified as part of the solution phase nucleic acid generation protocol, where the amplification may or may not occur prior to any fragmentation step. In those embodiments where the produced collection of nucleic acids has substantially the same complexity as the initial genomic source from which it is prepared, the amplification step employed is one that does not reduce the complexity, e.g., one that employs a set of random primers, as described below. For example, the initial genomic source may first be amplified in a manner that results in an amplified version of virtually the whole genome, if not the whole genome, before labeling, where the fragmentation, if employed, may be performed pre- or post-amplification.

In certain embodiments, the sample (or target composition) is labeled to make a population of labeled test target nucleic acids. In general, a target composition may be labeled using methods that are well known in the art (e.g., primer extension, random-priming, nick translation, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.), and, accordingly, such methods do not need to be described here in great detail. In certain of these embodiments, the test composition is labeled with a fluorescent label, which labels will be described in greater detail below.

In certain embodiments, a reference composition is employed. A reference composition may contain genomic material from any cell of an organism with a genome e.g., yeast, plants and animals, such as fish, birds, reptiles, amphibians and mammals. In certain embodiments, reference compositions containing genomic material from mice, rabbits, primates, or humans, etc, can be made and used. Suitable cells that may be used as a source of genomic material for use as reference compositions include: monkey kidney cells (COS cells), human embryonic kidney cells (HEK-293, Graham et al. J. Gen Virol. 36:59 (1977)); baby hamster kidney cells (BHK, ATCC CCL 10); chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (WI 38, ATCC CCL 75); human liver cells (hep G2, HB 8065); mouse mammary tumor (MMT 060562, ATCC CCL 51); TRI cells (Mather et al., Annals N. Y. Acad. Sci 383:44-68 (1982)); NIH/3T3 cells (ATCC CRL-1658); and mouse L cells (ATCC CCL-1). Additional cells (e.g. human lymphocytes) and cell lines will become apparent to those of ordinary skill in the art, and a wide variety of cell lines are available from the American Type Culture Collection, 10801 University Boulevard, Manassas, Va. 20110-2209.

In certain embodiments where two or more nucleic acid compositions, such as sample and reference compositions are employed, the compositions (or amplification products thereof), are distinguishably labeled using methods that are well known in the art (e.g., primer extension, random-priming, nick translation, end-labeling, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.).

The compositions may be labeled using “distinguishable” labels in that the labels can be independently detected and measured, even when the labels are mixed. In other words, the amounts of label present (e.g., the amount of fluorescence) for each of the labels are separately determinable, even when the labels are co-located (e.g., in the same tube or in the same duplex molecule or in the same feature of an array). Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), fluorescein and Texas red (Dupont, Boston Mass.), and POPRO3 and TOPRO3 (Molecular Probes, Eugene, Oreg.). Further suitable distinguishable detectable labels may be found in Kricka et al. (Ann Clin Biochem. 39:114-29, 2002).

In these embodiments, the labeling reactions produce first and second populations of labeled nucleic acids that correspond to the sample (i.e. test) and reference nucleic acid compositions, respectively. After nucleic acid purification and any pre-hybridization steps to suppress repetitive sequences (e.g., hybridization with Cot-1 DNA), the populations of labeled nucleic acids are contacted to an array of surface bound nucleic acids, as discussed above, under conditions such that nucleic acid hybridization to the surface bound polynucleotides can occur, e.g., in a buffer containing 50% formamide, 5×SSC and 1% SDS at 42° C., or in a buffer containing 5×SSC and 1% SDS at 65° C., both with a wash of 0.2×SSC and 0.1% SDS at 65° C.

In certain embodiments, the test and/or reference samples have non-reduced complexity as compared to the initial genomic sample. A non-reduced complexity target composition is one that is produced in a manner designed to not reduce its complexity. A target composition is considered to be a non-reduced complexity product composition as compared to the initial nucleic acid source (e.g., genomic source) from which it is prepared if there is a high probability that a sequence of specific length randomly chosen from the sequence of the initial genomic source is present in the product composition, either in a single nucleic acid member of the product or in a “concatamer” of two different nucleic acid members of the product (i.e., in a virtual molecule produced by joining two different members to produce a single molecule). A more detailed description of non-reduced complexity target compositions is presented in U.S. application Ser. No. 10/828,986 filed Apr. 20, 2004 to Barrett et al., published as 20050233340 on Oct. 20, 2005), which is incorporated herein by reference.

A non-reduced complexity target composition can be readily identified using a number of different protocols. One convenient protocol for determining whether a given collection of nucleic acids is a non-reduced complexity collection of nucleic acids is to screen the collection using a genome wide array of features for the initial, e.g., genomic source of interest. Thus, one can tell whether a given target composition has non-reduced complexity with respect to its genomic source by assaying the composition with a genome wide array for the genomic source. The genome wide array of the genomic source for this purpose is an array of features in which the collection of features of the array used to test the sample is made up of sequences uniformly and independently randomly chosen from the initial genomic source. As such, sequences of a particular length independently chosen randomly from the initial genomic source that uniformly sample the initial genomic source are present in the collection of features on the array. By uniformly is meant that no bias is present in the selection of sequences from the initial genomic source. In such a genome wide assay of sample, a non-reduced complexity sample is one in which substantially all of the array features on the array specifically hybridize to nucleic acids present in the sample, where by substantially all is meant at least about 10%, for example at least about 25%, including at least about 50%, such as at least about 60, 70, 75, 80, 85, 90 or 95% or more.

As such, according to the above guidelines, a sample is considered to be of non-reduced complexity as compared to its genomic source if its complexity is at least about 10%, for example at least about 25%, including at least about 50%, such as at least about 60, 70, 75, 80, 85, 90 or 95% or more of the complexity of the genomic source.

In certain other embodiments, the test and/or reference compositions may be of reduced complexity as compared to the initial genomic source. By reduced complexity is meant that the complexity of the target composition is at least about 20-fold less, such as at least about 25-fold less, at least about 50-fold less, at least about 75-fold less, at least about 90-fold less, at least about 95-fold less complex, than the complexity of the initial genomic source in terms of total numbers of sequences found in the test and reference compositions as compared to the initial genomic source. Examples of protocols that can produce reduced complexity product compositions of utility in genotyping and gene expression include those described in U.S. Pat. No. 6,465,182 and published PCT application WO 99/23256; as well as published U.S. Patent Application No. 2003/0036069 and Jordan et al., Proc. Nat'l Acad. Sci. USA (Mar. 5, 2002) 99: 2942-2947. In each of these protocols that produce a reduced complexity product, primers are employed that have been designed to knowingly produce product nucleic acids from only a select fraction or portion of the initial genomic source, e.g., genome, where fraction or portion may be defined as a subset or representative subset of a genome.

The labeled nucleic acids can be contacted to an array(s) serially, or, in other embodiments, simultaneously (i.e., the labeled nucleic acids are mixed prior to their contacting with the array). Depending on how the nucleic acid populations are labeled (e.g., if they are distinguishably or indistinguishably labeled), the populations may be contacted with the same array or different arrays. Where the populations are contacted with different arrays, the different arrays are substantially, if not completely, identical to each other in terms of target feature content and organization in certain embodiments.

Standard hybridization techniques (using high stringency hybridization conditions) are employed in representative embodiments. Suitable methods are described in references describing CGH techniques (Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985). See also U.S. Pat. Nos: 6,335,167; 6,197,501; 5,830,645; and 5,665,549; the disclosures of which are herein incorporate by reference.

In representative embodiments, comparative genome hybridization methods comprise the following major steps: (1) immobilization of polynucleotides on a solid support; (2) pre-hybridization treatment to increase accessibility of support-bound polynucleotides and to reduce nonspecific binding; (3) hybridization of a mixture of labeled nucleic acids to the surface-bound nucleic acids, typically under high stringency conditions; (4) post-hybridization washes to remove nucleic acid fragments not bound to the solid support polynucleotides; and (5) detection of the hybridized labeled nucleic acids. The reagents used in each of these steps and their conditions for use vary depending on the particular application.

As indicated above, hybridization is carried out under suitable hybridization conditions, which may vary in stringency as desired. In certain embodiments, highly stringent hybridization conditions may be employed. The term “high stringent hybridization conditions” as used herein refers to conditions that are compatible to produce nucleic acid binding complexes on an array surface between complementary binding members, i.e., between the surface-bound polynucleotides and complementary labeled nucleic acids in a sample. Representative high stringency assay conditions that may be employed in these embodiments are provided above.

The above hybridization step may include agitation of the immobilized polynucleotides and the sample of labeled nucleic acids, where the agitation may be accomplished using any convenient protocol, e.g., shaking, rotating, spinning, and the like.

Following hybridization, the array-surface bound polynucleotides are typically washed to remove unbound labeled nucleic acids. Washing may be performed using any convenient washing protocol, where the washing conditions are typically stringent, as described above.

Following hybridization and washing, as described above, the hybridization of the labeled nucleic acids to the targets is then detected using standard techniques so that the surface of immobilized targets, e.g., the array, is read. Reading of the resultant hybridized array may be accomplished by illuminating the array and reading the location and intensity of resulting fluorescence at each feature of the array to detect any binding complexes on the surface of the array. For example, a scanner may be used for this purpose, which is similar to the AGILENT MICROARRAY SCANNER available from Agilent Technologies, Palo Alto, Calif. Other suitable devices and methods are described in U.S. patent applications: Ser. No. 09/846125 “Reading Multi-Featured Arrays” by Dorsel et al.; and U.S. Pat. No. 6,406,849, which references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of nucleic acids, and are suitable for some embodiments.

Results from the reading or evaluating may be raw results (such as fluorescence intensity readings for each feature in one or more color channels) or may be processed results (such as those obtained by subtracting a background measurement, or by rejecting a reading for a feature which is below a predetermined threshold, normalizing the results, and/or forming conclusions based on the pattern read from the array (such as whether or not a particular target sequence may have been present in the sample, or whether or not a pattern indicates a particular condition of an organism from which the sample came).

In certain embodiments, results are assessed by determining a level of binding of the labeled sample to a feature specific for that genomic region. The term “level of binding” means any assessment of binding (e.g. a quantitative or qualitative, relative or absolute assessment) usually done, as is known in the art, by detecting signal (i.e., pixel brightness) from the label associated with the labeled nucleic acids. Since the level of binding of a labeled test target nucleic acid to a subject split-probe feature is proportional to the level of bound label, the level of binding of labeled sample nucleic acid is usually determined by assessing the amount of label associated with the feature.

In certain embodiments, the methods include evaluating binding of a given feature specific to two populations of target nucleic acids that are distinguishably labeled (i.e., a test and reference sample). In these embodiments, for a single subject split-probe oligonucleotide feature, the results obtained from hybridization with a test and reference sample can be compared, usually after normalization of the data. The results may be expressed using any convenient means, e.g., as a number or numerical ratio, etc. For example, the genomic copy number of a genomic region of interest in a test sample can be evaluated by comparing the level of binding to a specific split-probe of the test sample to a reference sample with known genomic copy number.

By “normalization” is meant that data corresponding to the two target compositions are globally normalized to each other, and/or normalized to data obtained from controls (e.g., internal controls produce data that are predicted to equal in value in all of the data groups). Normalization generally involves multiplying each numerical value for one data group by a value that allows the direct comparison of those amounts to amounts in a second data group. Several normalization strategies have been described (Quackenbush et al, Nat Genet. 32 Suppl: 496-501, 2002, Bilban et al., Curr Issues Mol Biol. 4:57-64, 2002, Finkelstein et al, Plant Mol Biol. 48(1-2):119-31, 2002, and Hegde et al, Biotechniques. 29:548-554, 2000). Specific examples of normalization suitable for use in the subject methods include linear normalization methods, non-linear normalization methods, e.g., using lowest local regression to paired data as a function of signal intensity, signal-dependent non-linear normalization, q-spline normalization and spatial normalization, as described in Workman et al., (Genome Biol. 2002 3, 1-16). In certain embodiments, the numerical value associated with a feature signal is converted into a log number, either before or after normalization occurs. Data may be normalized to data obtained from a support-bound polynucleotide for a genomic region of known copy number and/or methylation status in the target compositions.

In many embodiments, the assessment of the subject methods provides a numerical assessment of binding, and that numeral may correspond to an absolute level of binding, a relative level of binding, or a qualitative (e.g., presence or absence) or a quantitative level of binding. Accordingly, a binding assessment may be expressed as a ratio, whole number, or any fraction thereof. In other words, any binding may be expressed as the level of binding of a subject split-probe feature to a labeled test sample, divided by its level of binding to a labeled reference sample (or vice versa).

For example, in a methylation assessment embodiment described in greater detail below, a reference sample may be derived from the same genomic sample as was used to generate the test sample but was not contacted with the methylation sensitive restriction enzyme. In this example, if a ratio of test to reference sample binding is significantly below 1.0 (or is lower than an empirically derived value) for a particular subject oligonucleotide feature, the genomic region corresponding to that oligonucleotide is likely to be unmethylated (i.e., the genomic region is not methylated at the putative methylation site within the recognition sequence of the methylation-sensitive restriction enzyme contacted to the test sample).

While the embodiments described above are drawn to methods in which a single test and reference sample are employed, the subject invention is not limited to such embodiments. For example, in certain embodiments, multiple test samples can be prepared and assessed relative to a single reference sample, to multiple different reference samples, or to each other. As such, the categorization of which sample is called the test sample and which is called the reference sample depends on the specific implementation of the methods disclosed herein.

The subject methods described above find use in evaluating genomic regions from a variety of cells from bacteria, fungi, plants and animals, including insects, fish, birds, reptiles, amphibians and mammals. In certain embodiments, mammalian cells, i.e., cells from mice, rabbits, primates, or humans, or cultured derivatives thereof, may be used.

The above description is merely representative of ways of performing aCGH and is no way limiting.

### Utility

The subject split probe arrays and methods of using the same find use in a variety of different applications. One representative application in which the subject arrays and methods find use is the evaluation of the methylation status of one or more target genomic domains. In these representative embodiments, the split-probe nucleic acids of the present invention include two linked domains that flank a genomic domain that includes a genomic region of interest for which methylation status is to be evaluated (e.g., a 5′ domain and a 3′ domain). In embodiments in which the methylation status is to be evaluated, the genomic region of interest contains at least one putative methylation site present within the recognition sequence of a methylation-sensitive restriction enzyme (e.g., SmaI).

As shown in FIG. 1, target binding to the split-probe nucleic acid of the invention involves a “looping out” of the genomic domain (containing the genomic region of interest) that is not present in the split-probe (i.e., the intervening genomic region). In embodiments in which the methylation status is being evaluated, the genomic sample of interest is first contacted with a methylation-sensitive restriction endonuclease (i.e., one that cleaves DNA at the restriction site containing the putative methylation site) to generate the sample (i.e., target composition). If the putative methylation site is not methylated, the restriction site in the genomic region of interest is cleaved, resulting in the generation of a cleaved target which binds to the corresponding split-probe nucleic acids in a manner different from the intact target that is not cleaved, e.g., that exhibits “sub-optimal” binding to its cognate split-probe nucleic acid (see FIG. 1), as compared to the non-cleaved target. Conversely, if the putative methylation site is methylated, the restriction site will not be cleaved, resulting in the generation of a single intact target that exhibits optimal binding to its cognate split-probe nucleic acid (see FIG. 1), as compared to its cleaved counterpart. Accordingly, by evaluating the binding of the sample (test composition) (i.e., one that has been exposed to a methylation-sensitive restriction endonuclease) to a split-probe nucleic acid, the methylation status of the putative methylation site in the genomic region of interest is assessed.

The methods of the subject invention are applicable to evaluating the genomic methylation status of genomic samples from a wide variety of cells and cell types, including genomic samples derived from both prokaryotic and eukaryotic cells.

In embodiments in which genomic methylation is to be evaluated, a genomic sample is prepared and contacted with a methylation-sensitive restriction enzyme that only cleaves at unmethylated recognition sites (under conditions suitable for activity of that enzyme) to produce a target composition. Examples of methylation-sensitive restriction enzymes include, but are not limited to, BstUI, SmaI, SacII, EagI, MspI, HpaII, HhaI and BssHII which are suitable for use in the subject methods. These enzymes can be purchased from a variety of sources, e.g., Invitrogen (Carlsbad, Calif.) and Stratagene (La Jolla, Calif.), and conditions suitable for their activity are usually supplied with the enzyme when purchased. Accordingly, a genomic sample is contacted with a methylation-sensitive enzyme, and any unmethylated recognition sites in the genomic sample are cleaved. As indicated above, target compositions produced from methylation-sensitive restriction enzyme-exposed genomic samples are samples or test compositions. After contact with the restriction enzyme, the recognition sites present in the test target compositions may be cleaved, uncleaved, or a mixture thereof. In other words, if a sample contains a population of the same genomic region comprising the same methylation site, none, some or all of these sites may be methylated. Accordingly, target compositions made by contacting that sample with a methylation-sensitive restriction enzyme may contain genomic regions of interest that are intact, cleaved, or a mixture thereof.

In certain embodiments, the labeled sample is contacted with a subject split-probe oligonucleotide under conditions of stringency, usually high stringency, and any binding of the labeled targets in the sample to the split-probe nucleic acid is detected by detecting the label associated with the target. As mentioned above, in methylation status evaluation embodiments, binding to cognate split-probe oligonucleotides is optimal when the genomic region of interest is not cleaved (i.e., the target is intact) and sub-optimal when it is cleaved (the target is not intact). Therefore, optimal binding of a sample target to its cognate split-probe oligonucleotide indicates that the putative methylation site in the genomic region of interest is methylated, whereas sub-optimal binding indicates that the methylation site is not methylated. Whether a given binding observation is optimal or suboptimal can be readily determined by comparing the observed signal from a given feature to a suitable reference, e.g., a threshold intensity value above which binding is determined to be optimal, and below which binding is determined to be suboptimal.

In certain embodiments in which methylation status is being evaluated, a reference composition is also assayed, where the reference composition is made from the same genomic sample that was used to generate the test composition, as described above. Accordingly, in these embodiments, a genomic sample is prepared and used to make at least one test target composition and at least one reference target composition, usually from equal aliquots of the genomic sample. In certain of these embodiments, the reference target composition differs from the test target composition in that it is not contacted with the methylation-sensitive enzyme. In still other embodiments, the reference target composition differs from the test target composition in that it is contacted with a methylation-insensitive isoschizomer of the methylation-sensitive restriction enzyme used to make the test target composition. An isoschizomer of a restriction enzyme is one that has the same recognition sequence and cleaves the same site in the same manner. Although they cleave the same recognition sequences, isoschizomers may differ in their activity in the presence of various modifiers of DNA sequences such as methylation. Suitable methylation-insensitive isoschizomers are well known in the art, and include, e.g. MspI, a methylation-insensitive isoschizomer of HpaII, and XmaI a methylation-insensitive isoschizomer of SmaI.

The subject methods are suitable for simultaneous assessment of the copy number and/or methylation of a large number of genomic regions.

The subject split-probe nucleic acid arrays and methods for using the same may also be employed in other aCGH applications. The above-described arrays and methods find use in any application in which one wishes to compare the copy number of nucleic acid sequences found in two or more populations. One type of representative application in which the subject methods find use is the quantitative comparison of copy number of one nucleic acid sequence in a first collection of nucleic acid molecules relative to the copy number of the same sequence in a second collection. The subject methods find use in the detection of both heterozygous and homozygous deletions of sequences, as well as amplification of sequences, which conditions may be characteristic of certain conditions, e.g., disease conditions.

As such, embodiments of the present invention may be used in methods of comparing abnormal nucleic acid copy number and mapping of chromosomal abnormalities associated with disease. In certain embodiments, the subject methods are employed in applications that use nucleic acids immobilized on a solid support, to which differentially labeled solution phase nucleic acids produced as described above are hybridized. Analysis of processed results of the described hybridization experiments provides information about the relative copy number of nucleic acid domains, e.g. genes, in genomes.

Such applications compare the copy numbers of sequences capable of binding to the features. Variations in copy number detectable by the methods of the invention may arise in different ways. For example, copy number may be altered as a result of amplification or deletion of a chromosomal region, e.g. as commonly occurs in cancer.

Representative applications in which the subject methods find use are further described in U.S. Pat. Nos. 6,335,167; 6,197,501; 5,830,645; and 5,665,549; as well as published application serial nos. 20050064436; 20040248106; 20040241658; and 20040191813; the disclosures of which are herein incorporated by reference.

While the above-description has focused primarily comparative genomic hybridization applications, the invention is not so limited. As such embodiments of the invention include employing a split-probe array for use in assaying a variety of different types of nucleic acid samples, e.g., transcriptome (such as mRNA) samples, such as would be done in gene expression analysis applications; samples produced in location analysis or ChiP on chip applications (such as described in Published United States Application 20060035251, the disclosure of which is herein incorporated by reference, and the like.

### Computer-Related Embodiments

The invention also provides a variety of computer-related embodiments. For example, the designing of split-probes oligonucleotides can be done in silico using any number of probe design methods known to those of skill in the art. In this way, split-probes can be chosen that meet some basic criteria including characteristics of the intervening genomic domain (or region of interest therein), GC content, calculated melting temperatures and repetitive elements within the flanking sequences, to produce a sequence that is predicted to bind uniquely to the flanking domains of the intervening genomic domain.

In addition, the methods of analyzing data to assess genomic region copy number and/or methylation described in the previous section may be performed using a computer. Accordingly, the invention provides a computer-based system for assessing genomic region copy number and methylation using the above methods.

In certain embodiments, the design and analysis methods are coded onto a computer-readable medium in the form of “programming”, where the term “computer readable medium” as used herein refers to any storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer.

With respect to computer readable media, “permanent memory” refers to memory that is permanent. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination which will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

### Kits

Also provided by the subject invention are kits for practicing the subject methods, as described above. The subject kits at least include a split-probe nucleic acid, which in some embodiments, is present in an array of surface-bound nucleic acids. Other optional components of the kit include: a methylation-sensitive restriction enzyme, a methylation-insensitive isoschizomer of that enzyme, an enzyme that has a cleavage site generally outside of genomic region of interest, including the regions specific to the split-probe oligonucleotides, sample preparation reagents (e.g., to generate labeled test and/or reference samples), Cot-1 or other suppressors or repetitive DNA, and/or control or reference compositions for use in testing the other compositions of the kit. In some embodiments, arrays may be included in the kits. In alternative embodiments, the kit may also contain computer-readable media for performing the subject methods, as discussed above. The various components of the kit may be present in separate containers or certain compatible components may be precombined into a single container, as desired.

In addition to the above-mentioned components, the subject kits typically further include instructions for using the components of the kit to practice the subject methods (i.e., using the split-probe oligonucleotide in a method to evaluate the copy number and/or the methylation of a genomic region of interest). The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control analyte mixtures, e.g., two or more control compositions for use in testing the kit.

The following examples are offered by way of illustration and not by way of limitation.

### EXPERIMENTAL

Genomic regions where methylations occur, such as CpG islands, are often charcterized by high melting temperature (Tₘ) values and repetitive sequences for which good 60-mer probes are hard to find. Recently, we have shown that it is possible to design probes that consist of two somewhat shorter regions that have good probe characteristics that span an intervening genomic region that does not behave as well. These intervening genomic regions can span hundreds of nucleotides and contain multiple methylation sites. The following experiments demonstrate the value of this probe splitting strategy.

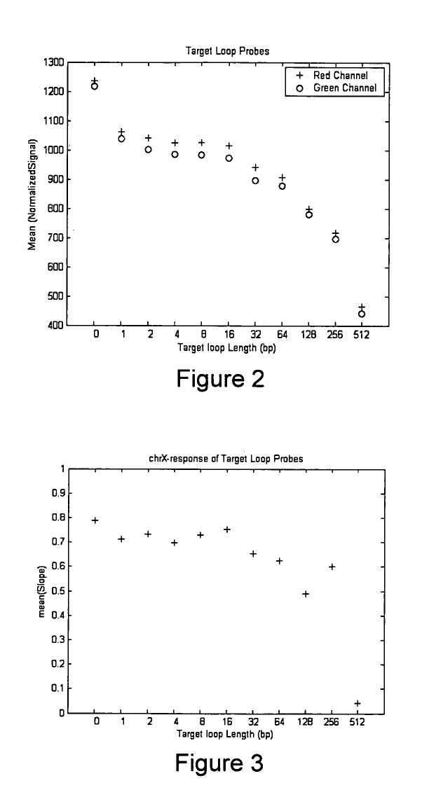

Test arrays were designed with a number of 60-mer test probes for CGH that comprise 5′ and 3′ domains (each containing 30 nucleotides). These probes were designed such that the 5′ and 3′ domains flanked 0, 1, 2, 4, 8, 16, 32, 64, 128, 256, or 512 nucleotides of genomic sequence (FIG. 2). A total of 3307 probes were designed covering every chromosome in the genome, with 10 to 12 probes per target sequence. All 369 targets were sufficiently long to provide intervals up to 128 bp, but the longer gaps are spanned by fewer targets sequences, and have fewer than 369 probes. These “split probes” consist of two sequences complementary to different domains on the target sequences of interest and are joined to form a single probe sequence.

Target sequences were selected to be devoid of repetitive sequences, as indicated by repeat-masker, and each contained a probe adjacent to the 5′-end of the target sequence and had good thermodynamic characteristics, i.e., the 60-mer probe starting at the 5′ end of the target has a duplex Tₘ close to 80° C. The remaining probes for the targets consisted of the combination of two 30-mers in genomic order, the 30-mer sequence at the 5′ end of the target is constant throughout all of the probes, whereas the 3′ 30-mer sequence for each probe starts at a position that varied from probe to probe (i.e., 0, 1, 2, 4, 8, 16, 32, 64, 128, 256, or 512 nucleotides of genomic sequence).

FIG. 2 shows the signal strength of the split-probes as a function of the length of the target loop on the test arrays. Genomic DNA from normal male (46,XY) and normal female (46, XX) samples were labeled with either Cy5 or Cy3 by nick translation using Klenow fragment of DNA polymerase I with a commercially available kit (Invitrogen, Carlsbad Calif.) according to the suppliers recommendation. Samples were combined after labeling then purified over MicroconY30 columns according to the suppliers recommendations. Human Cot1 DNA, yeast tRNA and tamra labeled 25mers were added to each sample with a final volume of 500 ul and a final 1× concentration of Agilent CGH hybridization buffer. Each sample was then boiled for 1.5 minutes and held at 37° C. prior to hybridization at 65° C., 40 RPM for 40 hours in a rotating oven. After hybridization, samples were washed 5 minutes with 0.5×SSPE buffer at room temperature then 1.5 minutes with 0.1×SSPE at 37° C. All arrays were then scanned using an Agilent scanner and default settings The signals shown in FIG. 2 are the normalized red and green signals. There is a signal decrease with increasing target loop length. About a factor of two reduction of signal is observed for target loop sizes up to about 250 bp. The signal decreases substantially after the introduction of the first base gap but is nearly the same for gaps from about 2 to 16 base pairs , then decreases more substantially after that. Therefore, for CGH measurements where up to 50% decreases in signals can be tolerated, target loops of up to 200 or more bases can be inserted without severe consequences. As such, the split-probes can be used as viable aCGH probes.

We next tested split-probes for their ability to perform in aCGH experiments with respect to sensitivity to copy number change. To do this, the split-probes were tested against standard aCGH (i.e., unsplit) probes in an aCGH assay using test and target compositions of known composition under the same conditions shown in FIG. 2. Specifically, hybridizations with 46,XY (male) and 46,XX (female) samples were used to calculate the slope response of all chromosome X probes on the arrays (256 probes, corresponding to 26 distinct targets). As shown in FIG. 3, the slopes of the split probes are diminished in comparison to the unsplit probes, but not significantly. The slopes drop immediately from a nominal (for this data of 0.80 to about 0.7 for Target loops from 1-16 bp and then decreases further for longer target loops). This performance is sufficient for making high-quality measurements of genomic copy number.

The above results and discussion demonstrate the usefulness of the split-probe oligonucleotide arrays of the present invention in aCGH assays. As such, split-probe oligonucleotide arrays enables the assessment if copy number and/or methylation status in genomic regions of a genome with sub optimal features (such as high melting temperature and GC content (i.e. >50% GC-content)).

The above-described compositions and methods find use in any application in which one wishes to assess copy number and/or methylation of a genomic region in a cell or sample of interest. One type of representative application in which the subject methods find use is the quantitative comparison of level of genome methylation (e.g., at CpG islands in a mammalian genome) in a first cell relative to the level of the same genome methylation in a second cell. Because the subject methods may be performed using a plurality of subject oligonucleotides in an array, the subject methods find most use in assessing global changes in methylation patterns between two cell or sample types.

The subject invention therefore finds use in methods for detecting differences in genome copy number and/or CpG methylation between two cells and, accordingly, finds particular use as a diagnostic and research tool for investigating a number of disease conditions as well as for assaying other cellular processes impacted by genome copy number variations and methylation.

As such, the subject methods represent a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of performing an array-based assay, said method comprising:
contacting an array comprising at least one split-probe nucleic acid feature specific for a nucleic acid of interest with a sample; and
assessing any binding to said split-probe nucleic acid to perform said assay.

2. The method according to claim 1, wherein said sample is a genomic sample.

3. The method according to claim 2, wherein said assay is a comparative genomic hybridization (aCGH).

4. The method according to claim 3, wherein said split-probe nucleic acid comprises a first domain linked to a second domain, wherein said first domain and said second domain specifically bind to separate genomic regions that flank an intervening genomic domain comprising a genomic region of interest.

5. The method according to claim 4, wherein said split-probe nucleic acid is from about 15 to about 100 nucleotides in length.

6. The method according to claim 4, wherein said intervening genomic domain is from about 5 to about 1000 nucleotides in length.

7. The method according to claim 4, wherein said array comprises a set of distinct split-probe nucleic acid features all specific for said genomic region of interest.

8. The method according to claim 7, wherein each split-probe nucleic acid member of said set comprises either an identical first domain or an identical second domain.

9. The method according to claim 4, wherein said genomic region of interest comprises at least one putative methylation site present in a recognition sequence of a methylation-sensitive restriction endonuclease.

10. The method according to claim 9, wherein said at least one putative methylation site is a CpG site.

11. The method according to claim 10, wherein said CpG site is present in a CpG island.

12. The method according to claim 1, wherein said sample is labeled.

13. The method according to claim 1, wherein said sample is a non-reduced complexity target composition.

14. The method according to claim 1, wherein said sample is a reduced complexity target composition.

15. The method according to claim 9, wherein said sample is produced from a portion of a genomic sample contacted with said methylation-sensitive restriction endonuclease.

16. The method according to claim 15, wherein said assessing step comprises comparing binding of said sample to said split-probe nucleic acid to binding of a reference composition to said split-probe oligonucleotide.

17. The method according to claim 16, wherein said reference composition is produced from a portion of said genomic sample that has not been contacted with said methylation sensitive restriction endonuclease.

18. The method according to claim 16, wherein said reference composition is produced from a portion of said genomic sample contacted with a methylation-insensitive isoschizomer of said methylation-sensitive restriction endonuclease.

19. The method according to claim 15, wherein said assessing step comprises evaluating the methylation status of said at least one putative methylation site in said genomic sample.

20. An array comprising at least one split-probe nucleic acid feature, wherein said split-probe nucleic acid comprises a first domain linked to a second domain, wherein said first and said second domains specifically bind to separate genomic regions that flank an intervening genomic domain comprising a genomic region of interest.

21. The array according to claim 20, wherein said split-probe nucleic acid is from about 15 to about 100 nucleotides in length.

22. The array according to claim 21, wherein said intervening genomic domain is from about 5 to about 1000 nucleotides in length.

23. The array according to claim 20, wherein said array comprises a set of distinct split-probe nucleic acid features all specific for said genomic region of interest.

24. The array according to claim 23, wherein each split-probe nucleic acid member of said set comprises either an identical first domain or an identical second domain.

25. The array according to claim 20, wherein said genomic region of interest comprises at least one putative methylation site present in a recognition sequence of a methylation-sensitive restriction endonuclease.

26. The array according to claim 25, wherein said at least one putative methylation site is a CpG site.

27. The array according to claim 26, wherein said CpG site is present in a CpG island.

28. A kit comprising:
an array comprising at least one split-probe nucleic acid specific for a genomic region of interest; and
instructions for using said array in an aCGH assay.

29. The kit according to claim 28, further comprising reagents for preparing a sample for use in said aCGH assay.

30. The kit according to claim 29, wherein said reagents comprise a methylation-sensitive restriction endonuclease.
